(19) United States
(12) Patent Application Publication
(10) Pub. No.: US 2013/0123354 A1
(43) Pub. Date: May 16, 2013

(54) SOLUBLE GUANYLATE CYCLASE (SGC) MODULATORS FOR TREATMENT OF LIPID RELATED DISORDERS

(71) Applicant: Ironwood Pharmaceuticals, Inc., Cambrdige, MA (US)

(72) Inventors: Mark G. CURRIE, Sterling, MA (US); Daniel P. ZIMMER, Somerville, MA (US)

(73) Assignee: Ironwood Pharmaceuticals, Inc., Cambrdige, MA (US)

(21) Appl. No.: 13/736,291

(22) Filed: Jan. 8, 2013

Related U.S. Application Data
(63) Continuation of application No. 12/594,744, filed on Nov. 3, 2009, now abandoned.
(60) Provisional application No. 60/910,309, filed on Apr. 5, 2007.

(51) Int. Cl. A61K 31/34; A61K 31/04

(52) U.S. Cl. CPC A61K 31/34; A61K 31/04
USPC 514/470; 514/742

(57) ABSTRACT

Disclosed herein are novel compositions and methods for treating or preventing a variety of disorders and conditions associated with lipid metabolism. The methods generally include administering to a patient in need thereof a therapeutically effective amount of a pharmaceutical composition comprising one or more sGC modulators alone or in combination with one or more lipid altering agents and/or PDE inhibitors.

### TECHNICAL FIELD

The subject matter of this application relates to the use of soluble guanylate cyclase (sGC) modulators, and pharmaceutical formulations thereof used alone or in combination with one or more additional agents for treating lipid related disorders and associated conditions.

### BACKGROUND

Soluble guanylate cyclase (sGC) is a nitric oxide (NO) sensing haemprotein that has been described in many eukaryotes. In response to various stimuli sGC converts GTP into the 2^{nd} messenger cyclic cGMP. GC is a heterodimeric protein consisting of homologous alpha and beta subunits. Each subunit consists of an N-terminal domain which may bind haem-nitric oxide and/or oxygen, a central domain of unknown function, and a C-terminal consensus nucleotide cyclase domain. sGC can be activated via both nitric oxide (NO) dependent and independent manners. When NO binds to the haem prosthetic group in the beta subunit of sGC, catalysis is accelerated by 2-3 orders of magnitude.

Nitric oxide (NO) is one of the smallest known biologically active messenger molecules. It is a colorless gas with good water solubility. NO was originally described as the principal endothelium-derived relaxing factor (“EDRF”), but it is now known to serve a variety of functions throughout the body, both physiological and pathophysiological, and has been shown to be involved in many important biological events. In the body, NO is synthesized from arginine and oxygen by various nitric oxide synthase (NOS) enzymes and by sequential reduction of inorganic nitrate. Three distinct isoforms of NOS have been identified. The NO produced from inducible NOS (iNOS or NOS II) in activated macrophage cells acts as a cytotoxic agent in informal immune defense against microorganisms and tumor cells. Constitutive neuronal NOS (nNOS or NOS I) is involved in neurotransmission and long term potentiation. Constitutive endothelial NOS (eNOS or NOS III) regulates smooth muscle relaxation and blood pressure.

Among its diverse functions, NO has been implicated in neurotransmission, immune regulation, blood vessel dilatation, vascular smooth muscle relaxation, modulation of the hair cycle, penile erections, and inhibition of platelet aggregation. Accordingly, medical conditions in which administering an sGC modulator is beneficial include diseases or disorders related to cardiovascular, gastrointestinal, inflammatory, respiratory, central nervous system, neurodegenerative, psychiatric, blood pressure-associated, coronary artery related, cholesterol level-associated, arterial thrombotic associated, and sexual function. Specific diseases or disorders which may be prevented or treated by administering an sGC modulator include but are not limited to: heart failure, stroke, septic shock, NSAID-induced gastric disease, inflammatory bowel disease, ischemic renal disease, peptic ulcer, diabetes, pulmonary hypertension, sickle cell anemia, atherosclerosis, asthma, chronic obstructive pulmonary disease or disorder, dementia, epilepsy, neuroinflammatory disease or disorder, trauma, multiple sclerosis, erectile dysfunction, male and female sexual dysfunction, and an age-related disease or disorder.

The management of dyslipidemia, a major risk factor for coronary heart disease, is an important part of the National Cholesterol Education Program Adult Treatment Panel III guidelines. The reduction of low-density lipoprotein cholesterol as the primary target of treatment is recommended. Accordingly, various lipid altering agents are considered first-line drugs for attaining this goal. However, monotherapy may not always be optimal for patients with significant combined or mixed dyslipidemias (e.g., elevated low-density lipoprotein cholesterol plus hypertriglyceridemia) or with concomitant conditions that increase the patient's level of risk (e.g., type 2 diabetes or the metabolic syndrome). Accordingly, combination drug therapy, which utilizes complementary mechanisms of action to enhance reduction of one lipoprotein (such as LDL or VLDL) or to affect two or more lipoproteins (such as LDL+VLDL±HDL±lipoprotein[a]), may be preferable.

Inhibiting NOS activity elevates serum triglyceride and cholesterol and lowers hepatic fatty acid oxidation (Khedara et al. (1996) J Nutr. 126:2563-7). Studies of rodents lacking n-NOS suggest a role for NO in the regulation of lipid metabolism (Schild et al. (2006) FASEB J. 20:145-7. Consistent with this, administration of arginine (an endogenous NOS substrate) and derivatives thereof reduces blood cholesterol levels (U.S. Pat. No. 5,157,022).

Accordingly, it would be desirable to develop an effective treatment for management of lipid related disorders using sGC modulators that can be administered alone or in combination with one or more lipid altering agents or PDE inhibitors. Lipid altering agents encompass several classes of drugs that include HMG CoA reductase inhibitors or statins, fibric acid derivatives (fibrates), cholesterol-ester-transfer-protein (“CETP”) inhibitors, squalene synthase inhibitors, microsomal-triglyceride-transfer-protein (“MTTP”) inhibitors, cholesterol absorption inhibitors (“CAIs”), bile acid sequestrants, nicotinic acid, and probucol and derivatives thereof (e.g. AGI-1067). These drugs differ with respect to mechanism of action and to the degree and type of lipid lowering. Thus, the indications for a particular drug are influenced by the underlying lipid abnormality. Monotherapy with statins, which competitively inhibit the intracellular rate-limiting enzyme for cholesterol biosynthesis, and bile acid sequestrants, which reduce terminal ileal bile acid absorption, primarily lowers plasma low-density lipoprotein (LDL) cholesterol by enhancing hepatic LDL-receptor activity. Monotherapy with fibrates, which serve as ligands for peroxisome proliferator-activated receptor α, a transcription factor influencing gene expression in lipid metabolism, reduces plasma very-low-density lipoprotein (VLDL) cholesterol and triglycerides, and also increases high-density lipoprotein (HDL) cholesterol. Niacin monotherapy reduces hepatic triglyceride production and inhibits HDL catabolism.

### sGC Modulators

Agents that function as sGC modulators include but are not limited to: NO donors, eNOS transcriptional enhancers, haem-dependent sGC stimulators, haem-independent sGC activators and NOS substrates.

### NO Donors

NO donors are pharmacologically active substances that release NO in vivo or in vitro. There are different classes of NO donors, which include organic nitrates (e.g., nitroglycerin, isosorbides (e.g. isosorbide dinitrate, isosorbide mononitrate, isosorbide 5-mononitrate, isosorbide 2-mononitrate, CA Registry no. 16051-77-7), S-nitrosothiols, iron-nitrosyl complexes (e.g., sodium nitroprusside), sydnonimines, C-nitroso compounds, and secondary amine/NO complex ions.

Specific examples of some of the classes of NO donors named above include: Isosorbide (Dilatrate®-SR, Imdur®, Ismo®, Isordil®, Isordil® Titradose®, Monoket®), FK 409 (NOR-3); FR 144420 (NOR-4); 3-morpholinosydnonimine; Linsidomine chlorohydrate (“SIN-1”); S-nitroso-N-acetylpenicillamine (“SNAP”); AZD3582 (CINOD lead compound), NCX 4016, NCX 701, NCX 1022, HCT 1026, NCX 1015, NCX 950, NCX 1000, NCX 1020, AZD 4717, NCX 1510/NCX 1512, NCX 2216, and NCX 4040 (all available from NicOx S.A.), 5-nitrosoglutathione (GSNO), 5-nitrosoglutathione mono-ethyl-ester (GSNO-ester), 6-(2-hydroxy-1-methyl-nitrosohydrazino)-N-methyl-1-hexanamine (NOC-9) or diethylamine NONOate, S-nitrosothiol, a nitrite, a sydnonimine, a NONOate, a N-nitrosoamine, a N-hydroxyl nitrosamine, a nitrosimine, a diazetine dioxide, an oxatriazole 5-imine, an oxime, a hydroxylamine, a N-hydroxyguanidine, a hydroxyurea or a furoxan. Nitric oxide donors are also as disclosed in U.S. Pat. Nos. 5,155,137, 5,366,997, 5,405,919, 5,650,442, 5,700,830, 5,632,981, 6,290,981, 5,691,423 5,721,365, 5,714,511, 6,511,911, and 5,814,666, Chrysselis et al. (2002) J Med Chem. 45:5406-9 (such as NO donors 14 and 17), and Nitric Oxide Donors for Pharmaceutical and Biological Research, Eds: Peng George Wang, Tingwei Bill Cai, Naoyuki Taniguchi, Wiley, 2005.

NO donors have a nitrate functionality within the molecule, and a nitroso functional group is present in all of these compounds. Glyceryl trinitrate (also known as GTN, nitroglycerin, nitroglycerine, and trinitrogylcerin) is the nitrate ester of glycerol. In sodium nitroprusside (SNP) a molecule of nitric oxide is coordinated to iron metal forming the square bipyramidal complex. 3-Morpholinosydnonimine (SIN-1) is a zwitterionic compound formed by combination of a morpholine and a sydnonimine. S-nitroso-N-acetylpenicillamine (SNAP) is an N-acetylated amino acid derivative with a nitrosothiol functional group. Diethylenetriamine/NO (DETA/NO) is a compound of nitric oxide covalently linked to diethylenetriamine. NCX 4016 is an m-nitroxymethyl phenyl ester of acetyl salicyclic acid.

The amount and duration of NO release by the respective NO donors determines their pharmacological properties. In vivo, some compounds act rapidly, and the amount of NO released is relatively small. In others, such as NCX 4016 (NO aspirin), the effect is slow and lasts for hours. The route of administration (oral and parenteral) and the duration of release of NO also differ. NO is connected with a specific molecular target; by binding to iron in the haem group of sGC, it produces cyclic guanosine monophosphate (cGMP), which activates a cascade of cellular processes.

The classic nitrovasodilators, organic nitrate and nitrite esters, including nitroglycerin, amyl nitrite, isosorbide dinitrate, isosorbide 5-mononitrate, and nicorandil, have been used for many years in the treatment of cardiovascular diseases. Their principal action is vasorelaxation/vasodilation, mediated by guanylyl cyclase activation and by direct inhibition of nonspecific cation channels in vascular smooth muscle cells (VSMCs). As such, these agents represent the prototypical form of NO-replacement therapy. All of the organic nitrate esters are prodrugs requiring enzymatic metabolism to generate bioactive NO. The major enzyme system involved is located within microsomal membranes, has an estimated apparent molecular mass of 160 kDa, and manifests enhanced activity in the presence of reducing equivalents, especially thiols, which potentiate the action of organic nitrate esters. Although the enzyme has not been more specifically characterized, growing evidence suggests that the cytochrome P-450 system, in conjunction with NADPH and glutathione-S-transferase activities, is required for the linked metabolic processes of denitration and reduction of organic nitrate esters to authentic NO.

In medicine, nitroglycerin is used as a heart medication (under the trade names NITROSPAN® and NITROSTAT®). It is used as a medicine for angina pectoris (ischaemic heart disease) in tablets, ointment, solution for intravenous use, transdermal patches (TRANSDERM NITRO®, NITRO-DUR®), or sprays administered sublingually (NITROLINGUAL PUMP SPRAY®, NATISPRAY®). Other nitroglycerin formulations are sold under the tradenames NITRO-BID®, NITROGLYN®, NITRONG®, NITRO-TIME®, NITROGARD®, NITROBID OIN 2%®, NITROTAB®, NITROQUICK®, and NITROGLYCERIN SLOCAPS®. The main effects of nitroglycerin in episodes of angina pectoris are subsiding of chest pain, decrease of blood pressure, and increase of heart rate. These effects arise because nitroglycerin is converted to nitric oxide in the body, and nitric oxide is a natural vasodilator.

The limitations of this class of agents are well known and include potentially adverse hemodynamic effects, drug tolerance, lack of selectivity, and limited bioavailability. Infrequent exposure to high doses of nitroglycerin can cause severe headaches known as NG head. These headaches can be severe enough to incapacitate some people, however, humans develop a tolerance and addiction to nitroglycerin after long-term exposure. Withdrawal can be fatal. Withdrawal symptoms include headaches and heart problems, with re-exposure to nitroglycerin these symptoms may disappear. Notwithstanding these shortcomings, prudent use of these agents yet represents the mainstay of therapy for patients with a variety of diseases and disorders.

eNOS Transcriptional Enhancers

Endothelial NO synthase is subject to physiological and pathophysiological regulation both at the transcriptional and at the post-transcriptional level. Compounds which enhance eNOS transcription are described in WO 02/064146, WO 02/064545, WO 02/064546 and WO 02/064565, and corresponding patent documents such as US2003/0008915, US2003/0022935, US2003/0022939 and US2003/0055093 for example. Other eNOS transcriptional enhancers include those described in US20050101599 (e.g. 2,2-difluorobenzo[1,3]dioxol-5-carboxylic acid indan-2-ylamide, and 4-fluoro-N-(indan-2-yl)-benzamide), and Sanofi-Aventis compounds AVE3085 and AVE9488 (CA Registry NO. 916514-70-0; Schafer et al., Journal of Thrombosis and Haemostasis 2005; Volume 3, Supplement 1: abstract number P1487).

### Haem-Dependent sGC Stimulators

Evgenov et al. (2006) Nature Reviews-Drug Discovery 5:755-768 review a novel class of haem-dependent sGC-stimulators which share several characteristics including crucial dependency on the presence of the reduced prosthetic haem moiety and strong synergistic enzyme activation when combined with NO. Haem-dependent sGC stimulators include but are not limited to:

YC-1 (see patent publications EP667345 and DE 19744026)

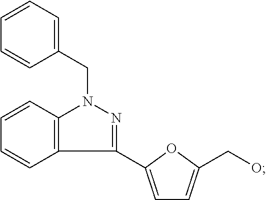

BAY 41-2272 (see patent publications DE19834047 and DE19942809)

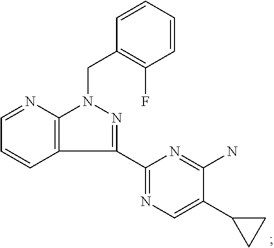 [SMILES: Nc1nc(-c2nn(Cc3ccccc3F)c3ncccc23)ncc1C1CC1]

BAY 41-8543 (see patent publication DE19834044)

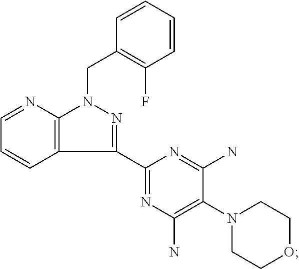 [SMILES: Nc1nc(-c2nn(Cc3ccccc3F)c3ncccc23)nc(N)c1N1CCOCC1]

CFM-1571 (see patent publication WO2000027394)

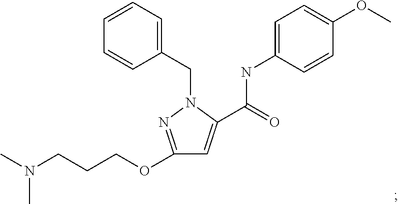

and

A350-619

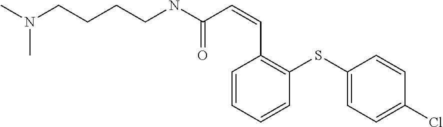 [SMILES: CN(C)CCCCNC(=O)/C=C\c1ccccc1Sc1ccc(Cl)cc1]

and other compounds disclosed in Tetrahedron Letters (2003), 44(48): 8661-8663.

### Haem-Independent sGC Activators

sGC can also be activated in a NO- and haem-independent manner by haem-independent sGC activators which include but are not limited to:

BAY 58-2667 (see patent publication DE19943635)

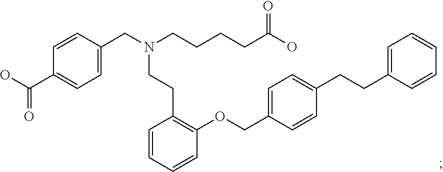 [SMILES: O=C(O)CCCCN(CCc1ccccc1OCc1ccc(CCc2ccccc2)cc1)Cc1ccc(C(=O)O)cc1]

HMR-1766 (ataciguat sodium, see patent publication WO2000002851)

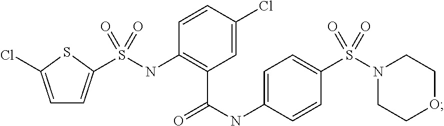 [SMILES: O=C(Nc1ccc(S(=O)(=O)N2CCOCC2)cc1)c1cc(Cl)ccc1NS(=O)(=O)c1ccc(Cl)s1]

S 3448 (2-(4-chloro-phenylsulfonylamino)-4,5-dimethoxy-N-(4-(thiomorpholine-4-sulfonyl)-phenyl)-benzamide; see patent publications DE19830430 and WO20000028511

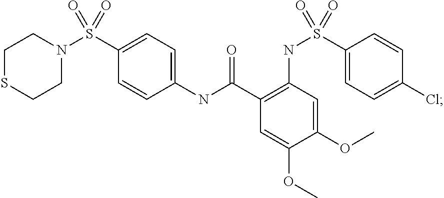 [SMILES: COc1cc(NS(=O)(=O)c2ccc(Cl)cc2)c(C(=O)Nc2ccc(S(=O)(=O)N3CCSCC3)cc2)cc1OC]

and

HMR-1069 (Sanofi-Aventis).

### NOS Substrates

L-arginine acts as the endogenous substrate of NOS. Other NOS substrates which can be converted to NO may be useful in the methods described herein. NOS substrates include n-hydroxyguanidine based analogs (such as N[G]-hydroxy-L-arginine (NOHA), (1-(3,4-dimethoxy-2-chlorobenzylideneamino)-3-hydroxyguanidine), and PR5 (1-(3,4-dimethoxy-2-chlorobenzylideneamino)-3-hydroxyguanidine); L-arginine derivatives (such as homo-Arg, homo-NOHA, N-tert-butyloxy- and N-(3-methyl-2-butenyl)oxy-L-arginine, canavanine, epsilon guanidine-carpoic acid, agmatine, hydroxyl-agmatine, and L-tyrosyl-L-arginine); N-alkyl-N′-hydroxyguanidines (such as N-cyclopropyl-N′-hydroxyguanidine and N-butyl-N′-hydroxyguanidine), N-aryl-N′-hydroxyguanidines (such as N-phenyl-N′-hydroxyguanidine and its para-substituted derivatives which bear —F, —Cl, -methyl, —OH substituents, respectively); guanidine derivatives such as 3-(trifluormethyl)propylguanidine; and others reviewed in Cali et al. (2005) Current Topics in Medicinal Chemistry 5:721-736) and disclosed in the references cited therein.

### SUMMARY

Briefly, the present application discloses treatment for various lipid related disorders in which the administration of a sGC modulator or pharmaceutical formulation thereof, used alone or in combination with one or more agents, is desirable.

In accordance with the above, the present application discloses methods to prevent and/or treat lipid related disorders such as dyslipidemia, hypercholesterolemia, hypertriglyceridemia, sitosterolemia, erectile dysfunction, fatty liver disease, and hepatitis by administering a therapeutically effective dose of at least one sGC modulator, alone or in combination with another therapeutic agent such as a lipid altering agent or a PDE inhibitor.

In a first aspect, methods for treating or preventing a lipid metabolism disorder by administering to a patient in need thereof a composition containing a therapeutically effective amount of one or more soluble guanylate cyclase modulators are disclosed.

In certain embodiments, the sGC modulator can include, for example, one or more of the following compounds: nitroglycerin, isosorbide dinitrate, isosorbide mononitrate, isosorbide 5-mononitrate, sodium nitroprusside, FK 409 (NOR-3); FR 144420 (NOR-4); 3-morpholinosydnonimine; Linsidomine chlorohydrate (“SIN-1”); S-nitroso-N-acetylpenicillamine (“SNAP”); AZD3582 (CINOD lead compound), NCX 4016, NCX 701, NCX 1022, HCT 1026, NCX 1015, NCX 950, NCX 1000, NCX 1020, AZD 4717, NCX 1510/NCX 1512, NCX 2216, and NCX 4040 (all available from NicOx S.A.), S-nitrosoglutathione (GSNO), S-nitrosoglutathione mono-ethyl-ester (GSNO-ester), 6-(2-hydroxy-1-methyl-nitrosohydrazino)-N-methyl-1-hexanamine (NOC-9) or diethylamine NONOate, S-nitrosothiol, a nitrite, a sydnonimine, a NONOate, a N-nitrosoamine, a N-hydroxyl nitrosamine, a nitrosimine, a diazetine dioxide, an oxatriazole 5-imine, an oxime, a hydroxylamine, a N-hydroxyguanidine, a hydroxyurea or a furoxan, including pharmaceutically acceptable salts or mixtures thereof.

In certain embodiments, the one or more sGC modulators are chosen from NO donors, eNOS transcriptional enhancers, haem-dependent sGC stimulators, haem-independent sGC activators and non-arginine NOS substrates. In certain embodiments, the NO donor is chosen from organic nitrates, isosorbides, S-nitrosothiols, iron-nitrosyl complexes, sydnonimines, C-nitroso compounds, and secondary amine/NO complex ions. In certain embodiments, the organic nitrates can include, for example nitroglycerin and isosorbides. Isosorbides include, but are not limited to, isosorbide dinitrate and isosorbide mononitrate. In certain embodiments, the isosorbide dinitrate can include, for example, dilatrate SR.

In other embodiments, the soluble guanylate cyclase modulator is an eNOS transcriptional enhancer. In certain embodiments, the eNOS transcriptional enhancer includes, but is not limited to, 2,2-difluorobenzo[1,3]dioxol-5-carboxylic acid indan-2-ylamide, 4-fluoro-N-(indan-2-yl)-benzamide), AVE3085 and AVE9488.

In other embodiments, the soluble guanylate cyclase modulator is haem-dependent sGC stimulator. In certain embodiments, the haem-dependent sGC stimulator includes, but is not limited to, YC-1, BAY 41-2272, BAY 41-8543, CFM-1571, and A350-619.

In other embodiments, the soluble guanylate cyclase modulator is haem-independent sGC activator. In certain embodiments, the haem-independent sGC activator includes, but is not limited to, BAY 58-2667, HMR-1766, S 3448 (2-(4-chloro-phenylsulfonylamino)-4,5-dimethoxy-N-(4-(thiomorpholine-4-sulfonyl)-phenyl)-benzamide and HMR-1069.

In other embodiments, the soluble guanylate cyclase modulator is a non-arginine NOS substrate. In certain embodiments, the non-arginine NOS substrate includes, but is not limited to, an n-hydroxyguanidine based analog, an L-arginine derivative, an N-alkyl-N′-hydroxyguanidine, an N-aryl-N′-hydroxyguanidine and a guanidine derivatives. In other embodiments, the non-arginine NOS substrate includes, but is not limited to, N[G]-hydroxy-L-arginine (NOHA), (1-(3,4-dimethoxy-2-chlorobenzylideneamino)-3-hydroxyguanidine), PR5 (1-(3,4-dimethoxy-2-chlorobenzylideneamino)-3-hydroxyguanidine), homo-Arg, homo-NOHA, N-tert-butyloxy-(3-methyl-2-butenyl)oxy-L-arginine, N-(3-methyl-2-butenyl)oxy-L-arginine, canavanine, epsilon guanidine-carpoic acid, agmatine, hydroxyl-agmatine, L-tyrosyl-L-arginine), N-cyclopropyl-N′-hydroxyguanidine, N-butyl-N′-hydroxyguanidine, N-phenyl-N′-hydroxyguanidine, a para-substituted derivative of N-phenyl-N′-hydroxyguanidine and 3-(trifluormethyl)propylguanidine.

In certain embodiments, the therapeutically effective amount of the pharmaceutical composition comprising one or more sGC modulators can be co-administered, either simultaneously or sequentially, with a therapeutically effective amount of at least one phosphodiesterase inhibitor and/or at least one lipid altering agent.

In certain embodiments, the phosphodiesterase inhibitor can include, for example, PDE3, PDE4, or PDE5. In some embodiments, the PDE5 inhibitor is chosen from sildenafil, tadalafil, vardenafil, udenafil and avanafil.

In other embodiments the lipid altering agent can include, for example, any compound having an indication for cholesterol absorption or metabolism, such as the following compounds: statins, fibrates, bile acid sequestrants, HMG-CoA reductase inhibitors, cholesterol absorption inhibitors, CETP inhibitors, MTTP inhibitors, and squalene synthetase inhibitors. Other suitable compounds are also disclosed herein. Suitable inorganic cholesterol sequestrants include bismuth salicylate plus montmorillonite clay, aluminum hydroxide and calcium carbonate antacids.

In certain embodiments, the lipid altering agent includes a fibrate, such as, for example, fenofibrate.

In other embodiments, the lipid altering agent includes a statin. In certain embodiments, the statin includes, but is not limited to, atorvastatin (Lipitor®), bervastatin, carvastatin, cerivastatin (Baycol®), crilvastatin, dalvastatin, fluvastatin (Lescol®), glenvastatin, fluindostatin, velostatin, lovastatin (mevinolin; Mevacor®), pravastatin (Pravachol®), rosuvastatin (Crestor®), and simvastatin (Zocor®).

In other embodiments, the lipid altering agent includes a bile acid sequestrant. In certain embodiments, the bile acid sequestrant includes, but is not limited to, cholestyramine, colesevelam, sevelamer, and colestipol.

In other embodiments, the lipid altering agent includes a cholesterol absorption inhibitor (CAI). In certain embodiments, the CAI includes, but is not limited to, 1,4-Diphenylazetidin-2-ones; 4-biarylyl-1-phenylazetidin-2-ones; 4-(hydroxyphenyl)azetidin-2-ones; 1,4-diphenyl-3-hydroxyalkyl-2-azetidinones; 4-biphenyl-1-phenylazetidin-2-ones; 4-biarylyl-1-phenylazetidin-2-ones; 4-biphenylylazetidinones. In other embodiments, the CAI includes, but is not limited to, (1S)-1,5-Anhydro-1-(4′-{(2S,3R)-3-[(3S)-3-(4-fluorophenyl)-3-hydroxypropyl]-4-oxo-1-phenylazetidin-2-yl}-3′-hydroxybiphenyl-4-yl)-D-glucitol, (3R,4S)-4-(3,3′-Dihydroxybiphenyl-4-yl)-3-[(3S)-3-(4-fluorophenyl)-3-hydroxypropyl]-1-phenylazetidin-2-one, and (4′-{(2S,3R)-3-[(3S)-3-(4-Fluorophenyl)-3-hydroxypropyl]-4-oxo-1-phenylazetidin-2-yl}-3′-hydroxybiphenyl-4-yl)phosphonic acid. In some embodiments, the CAI can be ezetimibe.

In certain embodiments, the pharmaceutical compositions described herein are administered to prevent and/or treat a lipid metabolism disorder chosen from dyslipidemia, hypercholesterolemia, hyperlipidemia, hypertriglyceridemia, sitosterolemia, and fatty liver disease. In certain embodiments, the hypercholesterolemia includes, for example, primary heterozygous familial hypercholesterolemia or primary non-familial hypercholesterolemia.

In certain embodiments, the pharmaceutical compositions described herein can be administered orally as sustained-release formulations.

In a second aspect, compositions comprising a therapeutically effective amount of at least one sGC modulator and at least one lipid altering agent are disclosed. In various embodiments, these compositions include, for example, substantially those embodiments described above.

In other embodiments, the compositions comprising a therapeutically effective amount of at least one sGC modulator and at least one lipid altering agent further comprise a therapeutically effective amount of at least one phosphodiesterase inhibitor.

In certain embodiments, the patient may be suffering from (or susceptible to developing) a lipid metabolism disorder including, but not limited to, dyslipidemia, hyperlipidemia, hypercholesterolemia, hypertriglyceridemia, sitosterolemia, familial hypercholesterolemia, xanthoma, combined hyperlipidemia, lecithin cholesterol acyltransferase deficiency, tangier disease, abetalipoproteinemia, erectile dysfunction, fatty liver disease, and hepatitis.

In certain embodiments, the pharmaceutical composition is in a form suitable for oral administration. In other embodiments the sGC modulator or pharmaceutical formulation is administered simultaneously with the lipid altering agent and/or PDE inhibitor. In yet other embodiments the sGC modulator or pharmaceutical formulation is administered sequentially to the lipid altering agent and/or PDE inhibitor.

Compositions containing a therapeutically effective amount of at least one sGC modulator and a therapeutically effective amount of at least one PDE inhibitor, wherein the compositions are useful for treating or preventing a lipid related disorder such as erectile dysfunction are also disclosed.

In certain embodiments a PDE inhibitor can include, for example, PDE3, PDE4, and PDE5. PDE5 includes, but is not limited to, any of the following compounds: sildenafil (VIAGRA®); vardenafil (LEVITRA®); tadalafil (CIALIS®), or any other inhibitor of an enzyme that accepts cGMP and breaks it down.

In yet another aspect, kits for treating a lipid metabolism disorder or associated condition comprising, in one or more containers, a therapeutically effective amount of the sGC modulator compositions as described in detail herein, and a label or packaging insert containing instructions for use are disclosed.

These, and other objects, features and advantages of this disclosure will become apparent from the following detailed description of the various aspects of the disclosure taken in conjunction with the accompanying Examples.

### DETAILED DESCRIPTION

The present application is based in part on the use of sGC modulators alone or in combination (for example, with one or more lipid altering agents or PDE inhibitors) to prevent/treat the lipid related disorders described herein.

The present application discloses compositions including at least one soluble guanylate cyclase modulator, either alone or in combination with at least one lipid altering agent, which when administered provide an effective treatment to patients suffering from, but not limited to, fatty liver disease, hepatitis, high serum levels of cholesterol, high serum levels of LDL, and high serum levels of triglycerides.

The present application also discloses compositions including at least one soluble guanylate cyclase modulator in combination with a PDE inhibitor, which when administered provide an effective treatment to patients suffering from a sexual dysfunction, such as erectile dysfunction.

Accordingly, the compositions disclosed herein are useful in methods for treating or preventing: a variety of lipid metabolism disorders and associated conditions such as, for example, hyperlipidemia, hypercholesterolemia, familial hypercholesterolemia, primary heterozygous familial hypercholesterolemia, primary non-familial hypercholesterolemia, xanthoma, combined hyperlipidemia, lecithin cholesterol acyltransferase deficiency, tangier disease, and abetalipoproteinemia; fatty liver disease; hepatitis; and erectile dysfunction.

Thus, the present application includes compositions comprising therapeutically effective amounts of at least one soluble guanylate cyclase modulator, or pharmaceutically acceptable salts thereof, alone or in combination with at least one lipid altering agent or PDE inhibitor, or pharmaceutically acceptable salts thereof.

The present application also includes methods for treating or preventing a variety of disorders by administering to a patient in need thereof a therapeutically effective amount of a pharmaceutical composition as disclosed and described in detail herein.

As employed above and throughout the disclosure, the following terms are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art unless otherwise indicated. As used herein and in the appended claims, the singular forms include plural referents unless the context clearly dictates otherwise. Thus, for example, reference to “a soluble guanylate cyclase modulator” includes one or more of such modulators, as would be known to those skilled in the art.

As used by those ordinarily skilled in the art, the term “Nitric Oxide Synthase substrates” or “NOS substrates” typically includes arginine, which is a known endogenous NOS substrate. To the extent that the present disclosure is directed to the use of sGC modulators such as NOS substrates to treat lipid related disorders such as high cholesterol, the appended claims are not intended to encompass the use of arginine to achieve such effect. Accordingly, as used herein, the term “non-arginine Nitric Oxide Synthase substrates” or “non-arginine NOS substrates” refers to substrates that are free of arginine, its salts and di- and tri-peptides containing arginine as disclosed by U.S. Pat. No. 5,157,022.

As used herein, “treating” or “treatment of” a condition or subject refers to taking steps to obtain beneficial or desired results, including clinical results. For purposes of this disclosure, beneficial or desired clinical results include, but are not limited to, alleviation or amelioration of one or more disease, symptom, or condition related to lipid metabolism disorders, fatty liver disease, hepatitis, or erectile dysfunction.

As used herein, a “therapeutically effective amount” of a drug or pharmaceutical composition or formulation, or agent, described herein is an amount of a drug or agent that, when administered to a subject with a disease or condition, will have the intended therapeutic effect, e.g., alleviation, amelioration, palliation or elimination of one or more manifestations of the disease or condition in the subject. The full therapeutic effect does not necessarily occur by administration of one dose and may occur only after administration of a series of doses. Thus, a therapeutically effective amount may be administered in one or more administrations.

As used herein, a “prophylactically effective amount” of a drug or pharmaceutical composition or formulation, or agent, described herein is an amount of a drug or agent that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose and may occur only after administration of a series of doses. Thus, a prophylactically effective amount may be administered in one or more administrations.

As used herein, and as would be understood by the person of skill in the art, the recitation of “a compound” or “a composition” is intended to include salts, solvates and inclusion complexes of that compound as well as any stereoisomeric form, or a mixture of any such forms of that compound in any ratio.

The term “pharmaceutically acceptable salt” refers to salts prepared from pharmaceutically acceptable non-toxic acids or bases including inorganic acids and bases and organic acids and bases. When the compounds of the present disclosure are basic, salts may be prepared from pharmaceutically acceptable non-toxic acids including inorganic and organic acids. Suitable pharmaceutically acceptable acid addition salts for the compounds of the present disclosure include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present disclosure include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine.

Administration of any of the compositions or formulations described in detail herein includes parallel administration (i.e., administration of elements of the formulation to the subject over a period-of time), co-administration or sequential administration (in which elements of the formulation are administered at approximately the same time, e.g., within about a few seconds to a few hours of one another), and simultaneous or co-formulation (in which elements of the formulation are combined or compounded into a single dosage form suitable for oral or parenteral administration).

Combination therapy can be achieved by administering two or more agents, e.g., a soluble guanylate cyclase modulator and a lipid altering agent or PDE inhibitor, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

A “subject” or “patient” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

A “susceptible individual” or “patient in need thereof” is an individual who suffers from, is suffering from, or is likely to or predisposed to suffer from a disorder or associated condition contemplated of being treated by the compositions described in detail herein. In humans these conditions may include, for example, dyslipidemia, hyperlipidemia, hypercholesterolemia, hypertriglyceridemia, sitosterolemia, erectile dysfunction, fatty liver disease, and hepatitis.

As used herein, the term “nitric oxide donor” is also interchangeably used herein and in the art with “NO prodrugs” or “NO-donating agents,” which refer to compounds that release free nitric oxide when administered to a patient, compounds that donate, release and/or directly or indirectly transfer a nitrogen monoxide species, and/or stimulate the endogenous production of nitric oxide or endothelium-derived relaxing factor (EDRF) in vivo and/or elevate endogenous levels of nitric oxide or EDRF in vivo and/or are oxidized to produce nitric oxide and/or are substrates for nitric oxide synthase and/or cytochrome P450.

The term “fibrate” is also interchangeably used herein and in the art with the term “fibric acid derivative,” and means any of the fibric acid derivatives useful in the methods described herein, e.g., fenofibrate. Fenofibrate is a fibrate compound, other examples of which include, for example, bezafibrate, beclofibrate, benzafibrate, binifibrate, ciprofibrate, clinofibrate, clofibrate, etofibrate, gemcabene, gemfibrozil, lifibrol, nicofibrate, pirifibrate, ronifibrate, simfibrate, theofibrate, etc.

Soluble guanylate cyclase modulators (e.g. nitroglycerin) can be used for preventing and/or treating a “lipid related disorder” or “lipid metabolism disorder” including, for example: reducing blood plasma or serum concentrations of LDL cholesterol; reducing concentrations of cholesterol and cholesterol ester in the blood plasma or serum; reducing blood plasma or serum concentrations of apolipoprotein B; reducing blood plasma or serum concentrations of triglycerides; increasing blood plasma or serum concentrations of high density lipoprotein (HDL) cholesterol; increasing fecal excretion of cholesterol; inhibiting the absorption of or reducing plasma or tissue concentration of one or more sterols or stanols; preventing or treating sistoserolemia; preventing or treating vascular diseases/disorders and conditions (including but not limited to arteriosclerosis, atherosclerosis, cardiovascular disease, cerebrovascular disease, renovascular disease, mesenteric vascular disease, pulmonary vascular disease, ocular vascular disease and peripheral vascular disease), hyperlipidemia (including but not limited to hypercholesterolemia, hypertriglyceridemia, sitosterolemia), hypertension, angina, cardiac arrhythmias, congestive heart failure, and stroke; reducing the incidence of cardiovascular disease-related events; preventing or treating vascular conditions and associated thrombotic events; preventing or treating vascular inflammation; reducing blood plasma or serum concentrations of C-reactive protein; preventing, treating, or ameliorating symptoms of Alzheimer's Disease (AD); regulating production or levels of at least one amyloid β (Aβ) peptide; regulating the amount of ApoE isoform 4 in the bloodstream and/or brain; preventing or treating obesity; preventing or decreasing the incidence of xanthomas; preventing or minimizing muscular degeneration and related side effects associated with certain HMG-CoA reductase inhibitors (statins); preventing or treating diabetes and associated conditions; preventing or treating at least one autoimmune disorder; preventing or treating demyelination and associated disorders; preventing or treating cholesterol associated tumors; inhibiting the expression of at least one multiple (“multi”)-drug resistance gene or protein in an animal cell; enhancing the effectiveness of a chemotherapeutic agent in a subject having cancer; reversing a multi-drug resistance phenotype exhibited by an animal cell; and preventing or treating osteopenia disorders (bone loss disorders).

As used herein the term “lipid altering agent” or “dyslipidemia agent” refers to compounds including, but not limited to, bile acid sequestrants such as cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colesevelam hydrochloride (such as WELCHOL® Tablets (polyallylamine hydrochloride) cross-linked with epichlorohydrin and alkylated with 1-bromodecane and (6-bromohexyl)-trimethylammonium bromide) which are available from Sankyo), colestipol (a copolymer of diethylenetriamine and 1-chloro-2,3-epoxypropane, such as COLESTID® tablets which are available from Pharmacia), dialkylaminoalkyl derivatives of a cross-linked dextran, LOCHOLEST®, DEAE-Sephadex (SECHOLEX®, POLICEXIDE®), water soluble derivatives such as 3,3-ioene, N-(cycloalkyl)alkylamines and poliglusam, insoluble quaternized polystyrenes, saponins and mixtures thereof and those bile acid sequestrants disclosed in WO97/11345, WO98/57652, U.S. Pat. No. 3,692,895, and U.S. Pat. No. 5,703,188. Suitable inorganic cholesterol sequestrants include bismuth salicylate plus montmorillonite clay, aluminum hydroxide and calcium carbonate antacids.

HMG-CoA reductase inhibitors are dyslipidemic agents that can be used in therapeutic combinations with compounds described herein. Suitable HMG-CoA reductase inhibitors for use in therapeutic combination with a compounds described herein include: atorvastatin (LIPITOR®; disclosed in U.S. Pat. No. 4,681,893, U.S. Pat. No. 5,385,929 and U.S. Pat. No. 5,686,104), atorvastatin calcium (disclosed in U.S. Pat. No. 5,273,995), dihydrocompactin, (disclosed in U.S. Pat. No. 4,450,171), bervastatin (disclosed in U.S. Pat. No. 5,082,859), carvastatin, cerivastatin (BAYCOL®; disclosed in U.S. Pat. No. 5,006,530, U.S. Pat. No. 5,502,199, and U.S. Pat. No. 5,177,080), crilvastatin, dalvastatin (disclosed in EP738510A2), fluvastatin (LESCOL®; disclosed in U.S. Pat. No. 4,739,073 and US534772), glenvastatin, fluindostatin (disclosed in EP363934A1), velostatin (visinolin; disclosed in U.S. Pat. No. 4,448,784 and U.S. Pat. No. 4,450,171), lovastatin (mevinolin; MEVACOR® (Merck and Co.) and related compounds disclosed in U.S. Pat. No. 4,231,938), mevastatin (and related compound disclosed in U.S. Pat. No. 3,983,140), compactin (and related compounds disclosed in U.S. Pat. No. 4,804,770), pitavastatin (also known as NK-104, itavastatin, nisvastatin, nisbastatin disclosed in U.S. Pat. No. 5,102,888), pravastatin (PRAVACHOL® (Bristol Myers Squibb) and related compounds disclosed in U.S. Pat. No. 4,346,227), rivastatin (sodium 7-(4-fluorophenyl)-2,6-diisopropyl-5-methoxymethylpyridin-3-yl)-3,5-dihydroxy-6-heptanoate), rosuvastatin (CRESTOR®; also known as ZD-4522 disclosed in U.S. Pat. No. 5,260,440), atavastatin, visastatin, simvastatin (ZOCOR® (Merck and Co.) and related compounds as disclosed in U.S. Pat. No. 4,448,784 and U.S. Pat. No. 4,450,171), sirrivastatin, CI-981, compounds disclosed in WO03/033481, U.S. Pat. No. 4,231,938, U.S. Pat. No. 4,444,784, U.S. Pat. No. 4,647,576, U.S. Pat. No. 4,686,237, U.S. Pat. No. 4,499,289, U.S. Pat. No. 4,346,227, U.S. Pat. No. 5,753,675, U.S. Pat. No. 4,613,610, EP0221025, and EP491226, and optical or geometric isomers thereof; and nontoxic pharmaceutically acceptable salts, N-oxides, esters, quaternary ammonium salts, and prodrugs thereof. In HMG-CoA reductase inhibitors where an open-acid form can exist, salt and ester forms may preferably be formed from the open-acid, and all such forms are included within the meaning of the term “HMG-CoA reductase inhibitor” as used herein. Pharmaceutically acceptable salts with respect to the HMG-CoA reductase inhibitor includes non-toxic salts of the compounds which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenzim-idazole, diethylamine, piperazine, and tris(hydroxymethyl)aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Other dyslipidemic agents which can be used in therapeutic combination with a compound described herein include:

HMG-CoA synthase inhibitors such as L-659,699 ((E,E)-11-[3′R-(hydroxymethyl)-4′-oxo-2′R-oxetanyl]-3,5,7R-trimethyl-2,4-undecadienoic acid) and those disclosed in U.S. Pat. No. 5,120,729, U.S. Pat. No. 5,064,856, and U.S. Pat. No. 4,847,271;

cholesterol absorption inhibitors such as plant sterols, plant stanols and/or fatty acid esters of plant stanols such as sitostanol ester used in BENECOL® margarine, stanol esters, beta-sitosterol, and sterol glycosides such as tiqueside. Other cholesterol absorption inhibitors include 1,4-Diphenylazetidin-2-ones; 4-biarylyl-1-phenylazetidin-2-ones; 4-(hydroxyphenyl)azetidin-2-ones; 1,4-diphenyl-3-hydroxyalkyl-2-azetidinones; 4-biphenyl-1-phenylazetidin-2-ones; 4-biarylyl-1-phenylazetidin-2-ones; and 4-biphenylylazetidinones.

acyl coenzyme A-cholesterol acyl transferase (ACAT) inhibitors such as avasimibe (Current Opinion in Investigational Drugs. 3(9):291-297 (2003)), eflucimibe, HL-004, lecimibe, DuP-128, KY505, SMP 797, CL-277,082 (Clin Pharmacol Ther. 48(2):189-94 (1990)) and the like; and those disclosed in U.S. Pat. No. 5,510,379, WO96/26948 and WO96/10559;

CETP inhibitors such as JTT 705 identified as in Nature 406, (6792):203-7 (2000), torcetrapib (CP-529,414 described in US20030186952 and WO00/017164), CP 532,632, BAY63-2149, SC 591, SC 795, and the like including those described in Current Opinion in Investigational Drugs. 4(3):291-297 (2003) and those disclosed in J. Antibiot., 49(8): 815-816 (1996), and Bioorg. Med. Chem. Lett., 6:1951-1954 (1996) and patent publications U.S. Pat. No. 5,512,548, U.S. Pat. No. 6,147,090, WO99/20302, WO99/14204, WO99/41237, WO95/04755, WO96/15141, WO96/05227, WO038721, EP796846, EP818197, EP818448, DE19704244, DE19741051, DE19741399, DE197042437, DE19709125, DE19627430, DE19832159, DE19741400, JP 11049743, and JP 09059155;

squalene synthetase inhibitors such as squalestatin-1, TAK-475, and those disclosed in U.S. Pat. No. 4,871,721, U.S. Pat. No. 4,924,024, U.S. Pat. No. 5,712,396 (α-phosphono-sulfonates), Biller et al (1988) J. Med. Chem., 31:1869 (e.g. isoprenoid (phosphinyl-methyl)phosphonates), Biller et al (1996) Current Pharmaceutical Design, 2:1, P. Ortiz de Montellano et al (1977) J. Med. Chem. 20:243 (terpenoid pyrophosphates), Corey and Volante (1976) J. Am. Chem. Soc., 98:1291 (farnesyl diphosphate analog A and presqualene pyrophosphate (PSQ-PP) analogs), McClard et al (1987) J.A.C.S., 109:5544 (phosphinylphosphonates), Capson, T. L., PhD dissertation, June, 1987, Dept. Med. Chem. U of Utah, Abstract, Table of Contents, pp 16, 17, 40-43, 48-51, Summary, (cyclopropanes), Curr. Op. Ther. Patents (1993) 861, and patent publications EP0567026A1, EP0645378A1, EP0645377A1, EP0611749A1, EP0705607A2, EP0701725A1, and WO96/09827;

antioxidants such as probucol (and related compounds disclosed in U.S. Pat. No. 3,674,836), probucol derivatives such as AGI-1067 (and other derivatives disclosed in U.S. Pat. No. 6,121,319 and U.S. Pat. No. 6,147,250), tocopherol, ascorbic acid, β-carotene, selenium and vitamins such as vitamin B6 or vitamin B12 and pharmaceutically acceptable salts and esters thereof;

PPARα agonists such as those disclosed in U.S. Pat. No. 6,028,109 (fluorophenyl compounds), WO00/75103 (substituted phenylpropionic compounds), WO98/43081 and fibric acid derivatives (fibrates) such as beclofibrate, benzafibrate, bezafibrate (C.A.S. Registry No. 41859-67-0, see U.S. Pat. No. 3,781,328), binifibrate (C.A.S. Registry No. 69047-39-8, see BE884722), ciprofibrate (C.A.S. Registry No. 52214-84-3, see U.S. Pat. No. 3,948,973), clinofibrate (C.A.S. Registry No. 30299-08-2, see U.S. Pat. No. 3,716,583), clofibrate (such as ethyl 2-(p-chlorophenoxy)-2-methyl-propionate, e.g. Atromid-S® capsules (Wyeth-Ayerst), etofibrate, fenofibrate (such as Tricor® micronized fenofibrate ((2-[4-(4-chlorobenzoyl)phenoxy]-2-methyl-propanoic acid, 1-methylethyl ester; Abbott Laboratories) or Lipanthyl® micronized fenofibrate (Labortoire Founier, France)), gemcabene, gemfibrozil (such as 5-(2,5-dimethylphenoxy)-2,2-dimethylpentanoic acid, e.g. Lopid® tablets (Parke Davis)), lifibrol, GW 7647, BM 170744, LY518674 and those fibrate and fibrate acid derivatives disclosed in WO03/033456, WO03/033481, WO03/043997, WO03/048116, WO03/053974, WO03/059864, and WO03/05875;

FXR receptor modulators such as GW 4064, SR 103912, and the like;

LXR receptor modulators such as GW 3965, T9013137, and XTC0179628, and those disclosed in US20030125357, WO03/045382, WO03/053352, WO03/059874, and the like;

HM74 and HM74A (human HM74A is Genbank Accession No. AY148884 and rat HM74A is EMM_patAR098624) receptor agonists such as nicotinic acid (niacin) and derivatives thereof (e.g. compounds comprising a pyridine-3-carboxylate structure or a pyrazine-2-carboxylate structure, including acid forms, salts, esters, zwitterions and tautomers, where available) including but not limited to those disclosed in Wise et al (2003) J. Biol. Chem. 278: 9869 (e.g. 5-methylpyrazole-3-carboxylic acid and acifran (4,5-dihydro-5-methyl-4-oxo-5-phenyl-2-furan carboxylic acid pyradine-3-acetic acid)), as well as 5-methyl nicotinic acid, nicotinuric acid, niceritrol, nicofuranose, acipimox (5-methylpyrazine-2-carboxylic acid 4-oxide), Niaspan® (niacin extended-release tablets; Kos) and those which can be easily identified by one skilled in the art which bind to and agonize the HM74A or HM74 receptor (for example using the assays disclosed in Wise et al (2003) J. Biol. Chem 278:9869 (nicotine binding and [35S]-GTPγS binding assays), Soga et al (2003) Biochem. Biophys. Res. Comm. 303:364 (radiolabel binding assay using the HM74 receptor which could be adapted to the HM74A receptor), Tunaru et al (2003) Nature Medicine 9:352 (calcium mobilization assay using the HM74 receptor which could be adapted to the HM74A receptor) and U.S. Pat. No. 6,420,183 (FLIPR assays are described generally in and may be adapted to the HM74A or HM74 receptor);

renin angiotensin system inhibitors;

bile acid reabsorption inhibitors (bile acid reuptake inhibitors), such as BARI 1453, SC435, PHA384640, S8921, AZD7706, and the like;

PPARδ agonists (including partial agonists) such as GW 501516, and GW 590735, and those disclosed in U.S. Pat. No. 5,859,051 (acetophenols), WO03/024395, WO97/28149, WO01/79197, WO02/14291, WO02/46154, WO02/46176, WO02/076957, WO03/016291, WO03/033493, WO99/20275 (quinoline phenyl compounds), WO99/38845 (aryl compounds), WO00/63161 (1,4-disubstituted phenyl compounds), WO01/00579 (aryl compounds), WO01/12612 & WO01/12187 (benzoic acid compounds), and WO97/31907 (substituted 4-hydroxy-phenylalconic acid compound);

sterol biosynthesis inhibitors such as DMP-565;

triglyceride synthesis inhibitors;

microsomal triglyceride transport (MTTP) inhibitors, such as inplitapide, LAB687, and CP346086, AEGR 733, implitapide and the like;

HMG-CoA reductase gene expression inhibitors (e.g. compounds that decrease HMG-CoA reductase expression by affecting (e.g. blocking) transcription or translation of HMG-CoA reductase into protein or compounds that may be biotransformed into compounds that have the aforementioned attributes by one or more enzymes in the cholesterol biosynthetic cascade or may lead to the accumulation of an isoprene metabolite that has the aforementioned activities (such regulation is readily determined by those skilled in the art according to standard assays (Methods of Enzymology, 110:9-19 1985))) such as those disclosed in U.S. Pat. No. 5,041,432 (certain 15-substituted lanosterol derivatives) and E. I. Mercer (1993) Prog. Lip. Res. 32:357 (oxygenated sterols that suppress the biosynthesis of HMG-CoA reductase);

squalene epoxidase inhibitors such as NB-598 ((E)-N-ethyl-N-(6,6-dimethyl-2-hepten-4-y-nyl)-3-[(3,3′-bithiophen-5-yl)methoxy]benzene-methanamine hydrochloride);

low density lipoprotein (LDL) receptor inducers such as HOE-402 (an imidazolidinyl-pyrimidine derivative that directly stimulates LDL receptor activity, see Huettinger et al (1993) Arterioscler. Thromb. 13:1005);

platelet aggregation inhibitors;

5-LO or FLAP inhibitors;

PPAR modulators (including compounds that may have multiple functionality for activating various combinations of PPARα, PPARγ, and PPARδ) such as those disclosed in U.S. Pat. No. 6,008,237, U.S. Pat. No. 6,248,781, U.S. Pat. No. 6,166,049, WO00/12491, WO00/218355, WO00/23415, WO00/23416, WO00/23425, WO00/23442, WO00/23445, WO00/23451, WO00/236331, WO00/236332, WO00/238553, WO00/50392, WO00/53563, WO00/63153, WO00/63190, WO00/63196, WO00/63209, WO00/78312, WO00/78313, WO01/04351, WO01/14349, WO01/14350, WO01/16120, WO01/17994, WO01/21181, WO01/21578, WO01/25181, WO01/25225, WO01/25226, WO01/40192, WO01/79150, WO02/081428, WO02/100403, WO02/102780, WO02/79162, WO03/016265, WO03/033453, WO03/042194, WO03/043997, WO03/066581, WO97/25042, WO99/07357, WO99/11255, WO99/12534, WO99/15520, WO99/46232, and WO98/05331 (including GW2331 or (2-(4-[difluorophenyl]-1 heptylureido)ethyl]phenoxy)-2-methylbutyric));

niacin-bound chromium, as disclosed in WO03/039535;

substituted acid derivatives disclosed in WO03/040114;

apolipoprotein B inhibitors such as those disclosed in WO02/090347, WO02/28835, WO03/045921, WO03/047575;

Factor Xa modulators such as those disclosed in WO03/047517, WO03/047520, WO03/048081;

ileal bile acid transport (“IBAT”) inhibitors (or apical sodium co-dependent bile acid transport (“ASBT”) inhibitors) such as benzothiepines (including 1,2-benzothiazepines; 1,4-benzothiazepines; 1,5-benzothiazepines; 1,2,5-benzothiadiazepines);

PPARδ activators such as disclosed in WO01/00603 (thiazole and oxazole derivates (e.g. C.A.S. Registry No. 317318-32-4), WO97/28149 (fluoro, chloro and thio phenoxy phenylacetic), U.S. Pat. No. 5,093,365 (non-1-oxidizable fatty acid analogues), and WO99/04815. Tests showing the efficacy of the therapy and the rationale for the combination therapy with a dyslipidemic agent are presented in US20030069221 (where the dyslipidemic agents are called ‘cardiovascular agents’).

The compounds described herein can be used in therapeutic combination with one or more anti-diabetic agents, including but not limited to:

PPARγ agonists such as glitazones (e.g., balaglitazone, ciglitazone, darglitazone (CP-86325, Pfizer), englitazone (CP-68722, Pfizer), isaglitazone (MIT/J&J), MCC-555 (Mitsibishi disclosed in U.S. Pat. No. 5,594,016), pioglitazone (such as such as Actos™ pioglitazone; Takeda), rosiglitazone (Avandia™; Smith Kline Beecham), rosiglitazone maleate, troglitazone (Rezulin®, disclosed in U.S. Pat. No. 4,572,912), GL-262570 (Glaxo Welcome), BRL49653 (disclosed in WO98/05331), CLX-0921, 5-BTZD, GW-0207, LG-100641, JJT-501 (JPNT/P&U), L-895645 (Merck), R-119702 (Sankyo/Pfizer), N,N-2344 (Dr. Reddy/NN), YM-440 (Yamanouchi), LY-300512, LY-519818, R483 (Roche), T131 (Tularik), and the like and compounds disclosed in U.S. Pat. No. 5,994,554, WO97/10813, WO97/27857,WO97/28115,WO97/28137,WO97/27847, WO00/76488, WO03/000685,WO03/027112,WO03/035602, WO03/048130, WO03/055867, and pharmaceutically acceptable salts thereof;

biguanides such as metformin hydrochloride (N,N-dimethylimidodicarbonimidic diamide hydrochloride, such as Glucophage™, Bristol-Myers Squibb); metformin hydrochloride with glyburide, such as Glucovance™ Bristol-Myers Squibb); buformin (Imidodicarbonimidic diamide, N-butyl-); etoformine (1-Butyl-2-ethylbiguanide, Schering A. G.) and phenformin;

protein tyrosine phosphatase-1B (PTP-1B) inhibitors, such as A-401,674, KR 61639, OC-060062, OC-83839, OC-297962, MC52445, MC52453, ISIS 113715, and those disclosed in WO03/032916, WO03/032982, WO03/041729, WO03/055883, WO02/26707, WO02/26743, JP2002114768, and pharmaceutically acceptable salts and esters thereof;

sulfonylureas such as acetohexamide (e.g. Dymelor, Eli Lilly), carbutamide, chlorpropamide (e.g. Diabinese®, Pfizer), gliamilide (Pfizer), glibenclamide, gliclazide (e.g. Diamcron, Servier Canada Inc), glimepiride (e.g. disclosed in US437978, such as Amaryl™, Aventis), glipentide, glipizide (e.g. Glucotrol or Glucotrol XL Extended Release, Pfizer), gliquidone, glisolamide, glyburide, glibenclamide (e.g. Micronase or Glynase Prestab, Pharmacia & Upjohn and Diabeta, Aventis), tolazamide (e.g. Tolinase), and tolbutamide (e.g. Orinase), and pharmaceutically acceptable salts and esters thereof;

meglitinides such as repaglinide (e.g. Pranidin®, Novo Nordisk), KAD1229 (PF/Kissei), and nateglinide (e.g. Starlix®, Novartis), and pharmaceutically acceptable salts and esters thereof;

alpha glucoside hydrolase inhibitors (or glucoside inhibitors) such as acarbose (e.g. Precose™, Bayer disclosed in U.S. Pat. No. 4,904,769), miglitol (such as GLYSET™, Pharmacia & Upjohn disclosed in U.S. Pat. No. 4,639,436), camiglibose (Methyl 6-deoxy-6-[(2R,3R,4R,5S)-3,4,5-trihydroxy-2-(hydroxymethyl)piperidino]-α-D-glucopyranoside, Marion Merrell Dow), voglibose (Takeda), adiposine, emiglitate, pradimicin-Q, salbostatin, CKD-711, MDL-25,637, MDL-73,945, and MOR 14, and the compounds disclosed in U.S. Pat. No. 4,062,950, U.S. Pat. No. 4,174,439, U.S. Pat. No. 4,254,256, U.S. Pat. No. 4,701,559, U.S. Pat. No. 4,639,436, U.S. Pat. No. 5,192,772, U.S. Pat. No. 4,634,765, U.S. Pat. No. 5,157,116, U.S. Pat. No. 5,504,078, U.S. Pat. No. 5,091,418, U.S. Pat. No. 5,217,877, US51091 and WO01/47528 (polyamines);

α-amylase inhibitors such as tendamistat, trestatin, and A1-3688, and the compounds disclosed in U.S. Pat. No. 4,451,455, U.S. Pat. No. 4,623,714, and U.S. Pat. No. 4,273,765;

insulin secreatagogues such as linogliride and A-4166 and pharmaceutically acceptable salts and esters thereof;

fatty acid oxidation inhibitors, such as clomoxir, and etomoxir, and pharmaceutically acceptable salts and esters thereof;

A2 antagonists, such as midaglizole, isaglidole, deriglidole, idazoxan, earoxan, and fluparoxan, and pharmaceutically acceptable salts and esters thereof;

insulin and related compounds (e.g. insulin mimetics) such as biota, LP-100, novarapid, insulin detemir, insulin lispro, insulin glargine, insulin zinc suspension (lente and ultralente), Lys-Pro insulin, GLP-1 (1-36) amide, GLP-1 (73-7) (insulintropin, disclosed in U.S. Pat. No. 5,614,492), LY-315902 (Lilly), GLP-1 (7-36)-NH2), AL-401 (Autoimmune), certain compositions as disclosed in U.S. Pat. No. 4,579,730, U.S. Pat. No. 4,849,405, U.S. Pat. No. 4,963,526, U.S. Pat. No. 5,642,868, U.S. Pat. No. 5,763,396, U.S. Pat. No. 5,824,638, U.S. Pat. No. 5,843,866, U.S. Pat. No. 6,153,632, U.S. Pat. No. 6,191,105, and WO 85/05029, and primate, rodent, or rabbit insulin including biologically active variants thereof including allelic variants, more preferably human insulin available in recombinant form (sources of human insulin include pharmaceutically acceptable and sterile formulations such as those available from Eli Lilly (Indianapolis, Ind. 46285) as Humulin™ (human insulin rDNA origin), also see the THE PHYSICIAN'S DESK REFERENCE, 55.sup.th Ed. (2001) Medical Economics, Thomson Healthcare (disclosing other suitable human insulins);

non-thiazolidinediones such as JT-501 and farglitazar (GW-2570/GI-262579), and pharmaceutically acceptable salts and esters thereof;

PPARα/γ dual agonists such as AR-H039242 (Aztrazeneca), GW-409544 (Glaxo-Wellcome), BVT-142, CLX-0940, GW-1536, GW-1929, GW-2433, KRP-297 (Kyorin Merck; 5-[(2,4-Dioxo thiazolidinyl)methyl]methoxy-N-[[4-(trifluoromethyl)phenyl]methyl]benzamide), L-796449, LR-90, MK-0767, SB 219994, muraglitazar, reglitazar (JTT-501) and those disclosed in WO99/16758, WO99/19313, WO99/20614, WO99/38850, WO00/23415, WO00/23417, WO00/23445, WO00/50414, WO01/00579, WO01/79150, WO02/062799, WO03/004458, WO03/016265, WO03/018010, WO03/033481, WO03/033450, WO03/033453, WO03/043985, WO 031053976 and pharmaceutically acceptable salts and esters thereof;

other insulin sensitizing drugs;

VPAC2 receptor agonists;

GLK modulators, such as those disclosed in WO03/015774;

retinoid modulators such as those disclosed in WO03/000249;

GSK 3β/GSK 3 inhibitors such as 4-[2-(2-bromophenyl)-4-(4-fluorophenyl-1H-imidazol-5-yl]pyridine and those compounds disclosed in WO03/024447, WO03/037869, WO03/037877, WO03/037891, WO03/068773, EP1295884, EP1295885, and the like;

glycogen phosphorylase (HGLPa) inhibitors such as CP-368,296, CP-316,819, BAYR3401, and compounds disclosed in WO01/94300, WO02/20530, WO03/037864, and pharmaceutically acceptable salts or esters thereof;

ATP consumption promotors such as those disclosed in WO03/007990;

TRB3 inhibitors;

vanilloid receptor ligands such as those disclosed in WO03/049702;

hypoglycemic agents such as those disclosed in WO03/015781 and WO03/040114;

glycogen synthase kinase 3 inhibitors such as those disclosed in WO03/035663;

agents such as those disclosed in WO99/51225, US20030134890, WO01/24786, and WO03/059870;

insulin-responsive DNA binding protein-1 (IRDBP-1) as disclosed in WO03/057827, and the like;

adenosine A2 antagonists such as those disclosed in WO03/035639, WO03/035640, and the like;

PPARδ agonists such as GW 501516, GW 590735, and compounds disclosed in JP10237049 and WO02/14291;

dipeptidyl peptidase IV (DP-IV) inhibitors, such as isoleucine thiazolidide, NVP-DPP728, P32/98, LAF 237, P3298, TSL225, valine pyrrolidide, TMC-2A/2B/2C, CD-26 inhibitors, FE999011, P9310/K364, VIP 0177, DPP4, SDZ 274-444, and the compounds disclosed in WO03/004498, WO03/004496, EP1258476, WO02/083128, WO02/062764, WO03/000250, WO03/002530, WO03/002531, WO03/002553, WO03/002593, WO03/000180, and WO03/000181;

GLP-1 agonists such as exendin-3 and exendin-4 (including the 39 aa peptide synthetic exendin-4 called Exenatide®), and compounds disclosed in US2003087821 and NZ 504256, and pharmaceutically acceptable salts and esters thereof;

peptides including amlintide and Symlin® (pramlintide acetate);

glycokinase activators such as those disclosed in US2002103199 (fused heteroaromatic compounds) and WO02/48106 (isoindolin-1-one-substituted propionamide compounds); and

other anti-diabetic agents such as cholestagel (Sankyo/Geltex), lipostabil (Rhone-Poulenc), Eisai E-5050 (an N-substituted ethanolamine derivative), imanixil (HOE-402), tetrahydrolipstatin (THL), istigmastanylphosphorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), acipimox, acifran, neomycin, p-aminosalicylic acid, aspirin, poly(diallylmethylamine) derivatives such as disclosed in U.S. Pat. No. 4,759,923, quaternary amine poly(diallyldimethylammonium chloride), pancreatic cholesteryl hydrolase (pCEH) inhibitors (such as WAY-121898), omega 3 fatty acids, fish oil (which contains Omega 3 fatty acids (3-PUFA)), and ionenes such as disclosed in U.S. Pat. No. 4,027,009. Tests showing the efficacy of the therapy and the rationale for the combination therapy with an anti-diabetic agent are presented in US20040214811.

Combination Therapy with PDE Inhibitors

The sGC modulators described herein can be used in combination therapy (for example in methods to treat a lipid related disorder or sexual (e.g. erectile) dysfunction) with one or more phosphodiesterase inhibitors. Phosphodiesterase (PDE) inhibitors slow the degradation of cyclic AMP (cAMP) and/or cyclic GMP (cGMP) by inhibition of the phosphodiesterases, which can lead to a relative increase in the intracellular concentration of cAMP and cGMP. High cholesterol is associated with erectile dysfunction (ED). The risk of ED is nearly two times greater in men with total cholesterol levels above 240 than in men with total cholesterol levels below 180. If there is too much cholesterol in the body, it sticks to the walls of the arteries. When cholesterol builds up, it creates a smaller opening for blood to pass through. This leads to less blood flow to the penis, which can create erectile problems. The physiologic mechanism of penile erection involves the release of nitric oxide and subsequent increased levels of cGMP which produce smooth muscle relaxation allowing increased blood flow. Thus, coadministration of sGC modulators and PDE inhibitors which slow the degradation of cGMP by phosphodiesterases ameliorates erectile dysfunction and is useful for treating lipid metabolism related disorders.

PDE inhibitors include PDE3 inhibitors, PDE4 inhibitors, PDE5 inhibitors, and inhibitors with multiple specificity including PDE3/4 and PDE3/4/5 inhibitors. Specific PDE inhibitors include those disclosed in patent publications DE1470341, DE2108438, DE2123328, DE2305339, DE2305575, DE2315801, DE2402908, DE2413935, DE2451417, DE2459090, DE2646469, DE2727481, DE2825048, DE2837161, DE2845220, DE2847621, DE2934747, DE3021792, DE3038166, DE3044568, EP000718, EP0008408, EP0010759, EP0059948, EP0075436, EP0096517, EP0112987, EP0116948, EP0150937, EP0158380, EP0161632, EP0161918, EP0167121, EP0199127, EP0220044, EP0247725, EP0258191, EP0272910, EP0272914, EP0294647, EP0300726, EP0335386, EP0357788, EP0389282, EP0406958, EP0426180, EP0428302, EP0435811, EP0470805, EP0482208, EP0490823, EP0506194, EP0511865, EP0527117, EP0626939, EP0664289, EP0671389, EP0685474, EP0685475, EP0685479, JP92234389, JP94329652, JP95010875, U.S. Pat. No. 4,963,561, U.S. Pat. No. 5,141,931, WO9117991, WO9200968, WO9212961, WO9307146, WO9315044, WO9315045, WO9318024, WO9319068, WO9319720, WO9319747, WO9319749, WO9319751, WO9325517, WO9402465, WO9406423, WO9412461, WO9420455, WO9422852, WO9425437, WO9427947, WO9500516, WO9501980, WO9503794, WO9504045, WO9504046, WO9505386, WO9508534, WO9509623, WO9509624, WO9509627, WO9509836, WO9514667, WO9514680, WO9514681, WO9517392, WO9517399, WO9519362, WO9522520, WO9524381, WO9527692, WO9528926, WO9535281, WO9535282, WO9600218, WO9601825, WO9602541, WO9611917, DE3142982, DE1116676, DE2162096, EP0293063, EP0463756, EP0482208, EP0579496, EP0667345 U.S. Pat. No. 6,331,543, US20050004222 (including those disclosed in formulas I-XIII and paragraphs 37-39, 85-0545 and 557-577), WO9307124, EP0163965, EP0393500, EP0510562, EP0553174, WO9501338 and WO9603399, as well as PDE5 inhibitors (such as RX-RA-69, SCH-51866, KT-734, vesnarinone, zaprinast, SKF-96231, ER-21355, BF/GP-385, NM-702, vardenafil (LEVITRA®); and tadalafil (CIALIS® and sildenafil (Viagra™)), PDE4 inhibitors (such as etazolate, ICI63197, RP73401, imazolidinone (RO-20-1724), MEM 1414 (R1533/R1500; Pharmacia Roche), denbufylline, rolipram, oxagrelate, nitraquazone, Y-590, DH-6471, SKF-94120, motapizone, lixazinone, indolidan, olprinone, atizoram, KS-506-G, dipamfylline, BMY-43351, atizoram, arofylline, filaminast, PDB-093, UCB-29646, CDP-840, SKF-107806, piclamilast, RS-17597, RS-25344-000, SB-207499, TIBENELAST, SB-210667, SB-211572, SB-211600, SB-212066, SB-212179, GW-3600, CDP-840, mopidamol, anagrelide, ibudilast, aminone, pimobendan, cilostazol, quazinone and N-(3,5-dichloropyrid-4-yl)-3-cyclopropylmethoxy-4-difluoromethoxybenzamide, PDE3 inhibitors (such as ICI153, 100, bemorandane (RWJ 22867), MCI-154, UD-CG 212, sulmazole, ampizone, cilostamide, carbazeran, piroximone, imazodan, CI-930, siguazodan, adibendan, saterinone, SKF-95654, SDZ-MKS-492, 349-U-85, emoradan, EMD-53998, EMD-57033, NSP-306, NSP-307, revizinone, NM-702, WIN-62582 and WIN-63291, enoximone and milrinone, PDE3/4 inhibitors (such as benafentrine, trequinsin, ORG-30029, zardaverine, L-686398, SDZ-ISQ-844, ORG-20241, EMD-54622, and tolafentrine) and other PDE inhibitors (such as vinpocetin, papaverine, enprofylline, cilomilast, fenoximone, pentoxifylline, roflumilast, and theophylline.

This present disclosure provides, in various embodiments, pharmaceutical combination kits and oral drug dosage forms that contain at least one soluble guanylate cyclase modulator and at least one lipid altering agent or phosophodiesterase inhibitor. In other embodiments, the present disclosure provides pharmaceutical combinations kits and oral dosage forms that contain at least one soluble guanylate cyclase modulator and at least one lipid altering agent and at least one phosophodiesterase inhibitor. The therapeutic agents may be contained in the same oral dosage form or in separate dosage forms that are administered sequentially. When more than two therapeutic agents are present in the pharmaceutical combination kit, all agents may be present in the same or different dosage forms and may be administered sequentially or simultaneously.

The active ingredients used in oral formulations, i.e., soluble guanylate cyclase modulators, either alone or in combination with one or more additional agents (e.g., lipid altering agents or PDE inhibitors), are well known in the art and many are commercially available. If desired, drugs can also be manufactured using methodology well known in the art.

### Formulation and Administration

The pharmaceutical compositions may include a “pharmaceutically acceptable inert carrier”, and this expression is intended to include one or more inert excipients, which include starches, polyols, granulating agents, microcrystalline cellulose, diluents, lubricants, binders, disintegrating agents, and the like. If desired, tablet dosages of the disclosed compositions may be coated by standard aqueous or nonaqueous techniques, “Pharmaceutically acceptable carrier” also encompasses controlled release means.

Compositions of the present invention may also optionally include other therapeutic ingredients, anti-caking agents, preservatives, sweetening agents, colorants, flavors, desiccants, plasticizers, dyes, and the like. Any such optional ingredient must, of course, be compatible with the compound of the invention to insure the stability of the formulation.

Examples of excipients for use as the pharmaceutically acceptable carriers and the pharmaceutically acceptable inert carriers and the aforementioned additional ingredients include, but are not limited to:

Binders: corn starch, potato starch, other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch (e.g., STARCH 1500® and STARCH 1500 LM®, sold by Colorcon, Ltd.), hydroxypropyl methyl cellulose, microcrystalline cellulose (e.g. AVICEL™, such as, AVICEL-PH-101™, -103™ and 105™, sold by FMC Corporation, Marcus Hook, Pa., USA), or mixtures thereof;

Fillers: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, or mixtures thereof;

Disintegrants: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof;

Lubricants: calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Degussa Co., Plano, Tex. USA), a pyrogenic silicon dioxide (CAB-O-SIL, Cabot Co., Boston, Mass. USA), or mixtures thereof;

Anti-caking agents: calcium silicate, magnesium silicate, silicon dioxide, colloidal silicon dioxide, talc, or mixtures thereof;

Antimicrobial agents: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenylmercuric acetate, phenylmercuric nitrate, potassium sorbate, propylparaben, sodium benzoate, sodium dehydroacetate, sodium propionate, sorbic acid, thimersol, thymo, or mixtures thereof; and

Coating agents: sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, carnuba wax, microcrystalline wax, or mixtures thereof.

It can be useful to administer a sGC modulator or pharmaceutical formulation described herein together with an HMG-CoA reductase inhibitor such as a statin, or with a PDE inhibitor. It can be particularly useful to combine a sGC modulator described herein together and an HMG-CoA reductase inhibitor such as a statin, or a PDE inhibitor, in a single pharmaceutical composition. The precise amount of each of these two active ingredients in a dosage unit will depend on the desired dosage of each component. Thus, it can be useful to create a dosage unit that will, when administered according to a particular dosage schedule (e.g., a dosage schedule specifying a certain number of units and a particular timing for administration), deliver the same dosage of each component as would be administered if the patient was being treated with only a single component. In other circumstances, it might be desirable to create a dosage unit that will deliver a dosage of one or both components that is less than that which would be administered if the patient was being treated only with a single component. Finally, it might be desirable to create a dosage unit that will deliver a dosage of one or both components that is greater than that which would be administered if the patient was being treated only with a single component. The pharmaceutical composition can include additional ingredients such as stabilizers or bulking agents.

Making of Pharmaceutical Preparations: The active agents used in the compositions of the present disclosure will typically be formulated in accordance with methods that are standard in the art (see e.g., Remington's Pharmaceutical Sciences, 16th edition, A. Oslow, editor, Easton, Pa. (1980)). Drugs may be prepared in admixture with conventional excipients, carriers, buffers, flavoring agents, etc. Typical carriers include, but are not limited to: water; salt solutions; alcohols; gum arabic; vegetable oils; benzyl alcohols; polyethylene glycols; gelatin; carbohydrates, such as lactose, amylose or starch; magnesium stearate; talc; silicic acid; paraffin; perfume oil; fatty acid esters; hydroxymethylcellulose; polyvinyl pyrrolidone; etc. Pharmaceutical preparations can be sterilized and, if desired, mixed with auxiliary agents such as: lubricants; preservatives; disintegrants; stabilizers such as cyclodextrans; wetting agents; emulsifiers; salts; buffers; natural or artificial coloring agents; natural or artificial flavoring agents; or aromatic substances. Pharmaceutical preparations can also include one or more of the following: acetylated monoglyceride, aspartame, beta carotene, calcium stearate, carnauba wax, cellulose acetate phthalate, citric acid, citric acid anhydrous, colloidal silicon dioxide, confectioner's sugar, crospovidone, docusate sodium, ethyl alcohol, ferric oxide, fructose, gelatin, glycerine, glyceryl monostearate (e.g. glyceryl monostearate 40-50), glyceryl triacetate, HPMC (hydroxypropyl methylcellulose), hydroxypropyl cellulose, hypromellose, iron oxide, isopropyl alcohol, lactose monohydrate, low substituted hydroxypropyl cellulose, magnesium carbonate, magnesium stearate, maltol, mannitol, methacrylic acid, methacrylic acid copolymer (e.g. methacrylic acid copolymer type C), methylcellulose, microcrystalline cellulose, mono ammonium glycyrrhizinate, n-butyl alcohol, paraffin, pectin propylene glycol alginate, polyacrylate, polyethylene glycol (e.g. polyethylene glycol 6000), polysorbate 80, polyvinyl pyrrolidone, povidone, propylene glycol, shellac, silicon dioxide, sodium carbonate, sodium citrate, sodium hydroxide, sodium lauryl sulfate, sodium stearyl fumarate, sorbitol, starch, sucrose, sugar sphere, talc, titanium dioxide, triethyl citrate, and xanthan gum. In certain embodiments, buffers that can raise the pH of the stomach are used. For example bicarbonate buffers may be included in the outer coating or as a rapidly dissolving, separate layer immediately below the outer coating.

The enteric coating surrounding the core may be applied using standard coating techniques. Materials used to form the enteric coating may be dissolved or dispersed in organic or aqueous solvents and may include one or more of the following: methacrylic acid copolymers; shellac; hydroxypropylmethylcellulose phthalate; polyvinyl acetate phthalate; hydroxypropylmethylcellulose trimellitate; carboxymethylcellulose; cellulose acetate phthalate; or other suitable enteric coating polymers. The pH at which the enteric coat will dissolve can be controlled by the polymer or combination of polymers selected and/or ratio of pendant groups. For example, dissolution characteristics of the coating can be altered by the ratio of free carboxyl groups to ester groups. Enteric coating layers may also contain pharmaceutical plasticizers such as: triethyl citrate; dibutyl phthalate; triacetin; polyethylene glycols; polysorbates; etc. Additives such as dispersants, colorants, anti-adhering and anti-foaming agents may also be included.

Making of Tablet Dosage Forms: Tablets can be made using standard technology well known in the art. Drugs used in the core or the outer coating may be granulated by methods such as slugging, low-shear or high-shear granulation, wet granulation, or fluidized bed granulation. Outer coatings may be formed by preparing a mixture containing appropriate polymers and a sufficient amount of drug to produce a therapeutically effective dose. The solution may then be sprayed on preformed, enterically-coated cores to produce the final tablets. If desired, a buffer layer or layer containing other agents may be interspersed between the enterically coated core and the outer coating.

In certain embodiments a pharmaceutical composition is prepared by adding a pharmaceutically acceptable carrier to the aforementioned compound, a pharmaceutically acceptable salt thereof, or a hydrate thereof as an active ingredient of the medicament of the present disclosure. As the medicament of the present disclosure, a substance, per se, that is selected from the group consisting of the alkylenedioxybenzene derivative and a pharmaceutically acceptable salt thereof, and a hydrate thereof and a solvate thereof may be administered to a mammal including human. In certain embodiments, pharmaceutical compositions comprising one or more of the aforementioned substances as an active ingredient and one or more of pharmaceutical additives are administered to a patient.

A variety of administration routes can be used in accordance with the present disclosure. An effective amount of the composition described herein can be administered parenterally, orally, by inhalation, nasally, buccally, or via an implanted reservoir. In certain embodiments the composition is administered orally. In certain embodiments oral sustained/extended release formulations are used.

Examples of the pharmaceutical composition include formulations for oral administration such as tablets, capsules, subtilized granules, powders, pills, troches, sublingual tablets and liquid preparations, and formulations for parenteral administration such as injections, suppositories, ointments, patches and the like.

In certain embodiments, formulations including those which slowly release the agent over time (i.e., sustained/extended release), such as found in lozenges, gums, and buccal patches are used. In other embodiments, formulations including agents in a bioadherent ingestible composition, such as those found in U.S. Pat. Nos. 5,858,391 and 5,670,163 to Cuca, et al. are used. The agent may also be formulated as a liquid or as a tablet, pill, capsule or powder to be dissolved in a liquid, and is preferably slowly sipped by the patient.

Tablets and capsules for oral administration are usually provided in a unit dosage form, and can be prepared by adding ordinary pharmaceutical carriers such as binders, fillers, diluents, compressing agents, lubricants, disintegrating agents, coloring matters, flavoring agents, and moistening agents. Tablets may be coated according to a well-known method, for example, by using an enteric coating agent. For example, fillers such as cellulose, mannitol and lactose; disintegrating agents such as starch, polyvinylpyrrolidone, starch derivatives and sodium starchglycolate; lubricants such as magnesium stearate; moistening agents such as sodium laurylsulfate and the like may be used.

Liquid preparations for oral administration can be provided in the forms of, for example, aqueous or oily suspensions, solutions, emulsions, syrups and elixirs, as well as dried formulations that are re-dissolvable before use by water or a suitable medium. Those liquid preparations may contain ordinary additives, for example, suspending agents such as sorbitol, syrups, methylcellulose, gelatin, hydroxyethylcellulose, carboxymethylcellulose, aluminum stearate gel and hydrogenated edible fats; emulsifiers such as lecitin, sorbitan monooleate and gum arabic; non-aqueous media including edible oils such as almond oil, rectified coconut oil, oily esters (e.g., esters of glycerin), propylene glycol and ethyl alcohol; preservatives such as methyl ester, ethyl ester and propyl ester of p-hydroxybenzoic acid and sorbic acid; and usual flavoring agents and coloring matters as required.

Formulations for oral administration can be manufactured according to a method well known in the art, for example, by mixing, filling, compressing and the like. In addition, it is also possible to disperse the active ingredient in a formulation containing a large amount of filler by repetitive mixing. Formulations for parenteral administration are generally provided as unit dosage form preparations containing the compound as the active ingredient and a sterilized medium. The solution for parenteral administration may generally be prepared by dissolving the compound in a medium, subjecting the resulting solution to filtration for sterilization, filling the solution in vials or ampoules, and sealing the vials or ampoules. It is also possible to freeze the composition and fill the result in vials, and then eliminate the moisture in vacuo to improve stability. Parenteral suspensions can be prepared by substantially the same method as that applied to solutions for parenteral administration; however, the suspensions can preferably be manufactured by suspending the active ingredient in a medium, and then subjecting the result to sterilization by using ethylene oxide or the like. Furthermore, surface active agents, moistening agents and so forth may also be added so that a uniform dispersion of the active ingredient can be obtained.

Combining two or more active ingredients in single dosage form results in the possibility of chemical interactions between the active drug substances. For example, acidic and basic active ingredients can react with each other and acidic active ingredients can facilitate the degradation of acid labile substances. Thus, in certain dosage forms, acidic and basic substances can be physically separated as two distinct or isolated layers in a compressed tablet, or in the core and shell of a press-coated tablet. Additional agents that are compatible with acidic as well as basic substances, have the flexibility of being placed in either layer. In certain multiple layer compositions at least one active ingredient can be enteric-coated. In certain embodiments thereof at least one active ingredient can be presented in a controlled release form. In certain embodiments where a combination of three or more active substances are used, they can be presented as physically isolated segments of a compressed multilayer tablet, which can be optionally film coated.

The therapeutic combinations described herein can be formulated as a tablet or capsule comprising a plurality of beads, granules, or pellets. All active ingredients including the vitamins of the combination are formulated into granules or beads or pellets that are further coated with a protective coat, an enteric coat, or a film coat to avoid the possible chemical interactions. Granulation and coating of granules or beads is done using techniques well known to a person skilled in the art. At least one active ingredient can present in a controlled release form. Finally these coated granules or beads are filled into hard gelatin capsules or compressed to form tablets.

The therapeutic combinations described herein can be formulated as a capsule comprising microtablets or minitablets of all active ingredients. Microtablets of the individual agents can be prepared using well known pharmaceutical procedures of tablet making like direct compression, dry granulation or wet granulation. Individual microtablets can be filled into hard gelatin capsules. A final dosage form may comprise one or more microtablets of each individual component. The microtablets may be film coated or enteric coated.

The therapeutic combinations described herein can be formulated as a capsule comprising one or more microtablets and powder, or one or more microtablets and granules or beads. In order to avoid interactions between drugs, some active ingredients of a said combination can be formulated as microtablets and the others filled into capsules as a powder, granules, or beads. The microtablets may be film coated or enteric coated. At least one active ingredient can be presented in controlled release form.

The therapeutic combinations described herein can be formulated wherein the active ingredients are distributed in the inner and outer phase of tablets. In an attempt to divide chemically incompatible components of proposed combination, few interacting components are converted in granules or beads using well-known pharmaceutical procedures in prior art. The prepared granules or beads (inner phase) are then mixed with outer phase comprising the remaining active ingredients and at least one pharmaceutically acceptable excipient. The mixture thus comprising inner and outer phase is compressed into tablets or molded into tablets. The granules or beads can be controlled release or immediate release beads or granules, and can further be coated using an enteric polymer in an aqueous or non-aqueous system, using methods and materials that are known in the art.

The therapeutic combinations described herein can be formulated as single dosage unit comprising suitable buffering agent. All powdered ingredients of said combination are mixed and a suitable quantity of one or more buffering agents is added to the blend to minimize possible interactions.

The agents described herein, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques. The agents described herein, alone or in combination, can be formulated using Nanocrystal® technology (Elan Corporation, Dublin, Ireland).

The agents can be a free acid or base, or a pharmacologically acceptable salt thereof. Solids can be dissolved or dispersed immediately prior to administration or earlier. In some circumstances the preparations include a preservative to prevent the growth of microorganisms. The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). The formulations may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Pharmaceutical agents can be sterilized by filter sterilization or by other suitable means.

Suitable pharmaceutical compositions in accordance with the invention will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences, 18th Ed., Mack Publishing Company, 1995.

The agent can be in the form of a pharmaceutically acceptable salt. Such salts are prepared from pharmaceutically acceptable non-toxic bases including inorganic bases and organic bases. Examples of salts derived from inorganic bases include aluminum, ammonium, calcium, copper, ferric, ferrous, lithium, magnesium, manganic salts, manganous, potassium, sodium, zinc, and the like. In some embodiments, the salt can be an ammonium, calcium, magnesium, potassium, or sodium salt. Examples of salts derived from pharmaceutically acceptable organic non-toxic bases include salts of primary, secondary, and tertiary amines, benethamine, N,N′-dibenzylethylenediamine, diethylamine, 2-diethylaminoethanol, 2-dimethylaminoethanol, diethanolamine, ethanolamine, ethylenediamine, N-ethylmorpholine, N-ethylpiperidine, epolamine, glucamine, glucosamine, histidine, hydrabamine, isopropylamine, lysine, methylglucamine, meglumine, morpholine, piperazine, piperidine, polyamine resins, procaine, purines, theobromine, triethylamine, trimethylamine, tripropylamine, and trolamine, tromethamine Examples of other salts include tris, arecoline, arginine, barium, betaine, bismuth, chloroprocaine, choline, clemizole, deanol, imidazole, and morpholineethanol.

The agents of the invention can be administered orally, e.g., as a tablet or cachet containing a predetermined amount of the active ingredient, pellet, gel, paste, syrup, bolus, electuary, slurry, capsule; powder; granules; as a solution or a suspension in an aqueous liquid or a non-aqueous liquid; as an oil-in-water liquid emulsion or a water-in-oil liquid emulsion, via a liposomal formulation (see, e.g., EP736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein.

### Dosing and Regimen

Doses of the aforementioned compound as the active ingredient can be suitably decided depending on the purpose of administration, i.e., therapeutic or preventive treatment, nature of a disease to be treated or prevented, conditions, body weight, age, sexuality and the like of a patient. In the method for administering the pharmaceutical preparation according to the present disclosure, the soluble guanylate cyclase modulator may be administered simultaneously with a lipid altering agent or PDE inhibitor, or the two may be sequentially administered in an optional order. Nitroglycerin, for example, comes as a sublingual tablet, buccal tablet, extended-release (long-acting) capsule, or spray to be used orally. The buccal extended-release tablets and the extended-release tablets and capsules are usually taken three to six times a day. The practically desirable method and sequence for administration varies depending on the purpose of administration, i.e., therapeutic or preventive treatment, nature of a disease to be treated or prevented, conditions, body weight, age, sexuality and the like of a patient. The optimum method and sequence for administration of the compounds described in detail herein under preset given conditions may be suitably selected by those skilled in the art with the aid of the routine technique and the information contained in the present specification and field of invention. In certain embodiments, an amount of about 0.05 mg to 20 mg, about 0.05 mg to 10 mg, about 0.01 mg to 3 mg, about 1 mg to 3 mg, about 0.1 mg to 1 mg, about 0.5 mg to 3 mg, about 2 mg to 3 mg, about 1 mg to 5 mg, or about 2 mg to 5 mg of a soluble guanylate cyclase modulator (such as nitroglycerin) per day for an adult can be orally administered in the formulation of a sublingual tablet, buccal tablet, extended-release (long-acting) capsule, or spray. In certain embodiments, an amount of about 10 mg to 120 mg, about 10 mg to 90 mg, about 30 mg to 60 mg, about 60 mg to 100 mg, or about 20 mg to 60 mg (e.g. 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg) of a soluble guanylate cyclase modulator (such as isosorbide mononitrate (e.g. isosorbide 5-mononitrate, isosorbide 2-mononitrate, CAS registry No. 16051-77-7) per day for an adult can be orally administered in the formulation of a sublingual tablet, buccal tablet, extended-release (long-acting) capsule, or spray. When administered in combination with a lipid altering agent where the lipid altering agent is a statin, about 2 mg to 80 mg, about 5 mg to 40 mg, or about 10 to 80 mg of a statin per day for an adult can be orally administered. When administered in combination with a lipid altering agent where the lipid altering agent is a bile acid sequestrant, about 1 g to 30 g, about 0.2 g to 6 g, about 0.1 g to 3 g, about 0.02 g to 0.6 g, about 0.01 g to 0.3 g, about 5 g to 150 g, about 2 g to 60 g or about 10 g to 300 g of a bile acid sequestrant (e.g. cholestyramine, colesevelam or colestipol) per day for an adult can be orally administered. When administered in combination with a lipid altering agent where the lipid altering agent is a fibrate about 5 mg to 150 mg of a fibrate (e.g. fenofibrate (Tricor®)) per day for an adult can be orally administered. When administered in combination with a lipid altering agent where the lipid altering agent is a cholesterol absorption inhibitor (e.g. ezetimibe), about 2 mg to 80 mg, about 5 mg to 40 mg, or about 10 to 80 mg of a cholesterol absorption inhibitor per day for an adult can be orally administered. When administered in combination with a PDE inhibitor, about 1 mg to 100 mg of a PDE inhibitor per day for an adult can be orally administered (e.g., 25 mg, 50 mg, or 100 mg of sildenafil; 2.5 mg, 5 mg, 10 mg, or 20 mg of vardenafil; or 5 mg, 10 mg, or 20 mg of tadalafil). Those of skill in the art would recognize that the dosing unit for the above agents can include any point inclusive of the range provided. Exemplary dosing units are provided in at least paragraphs [00116]-[00132] of the priority application U.S. Provisional Application No. 60/910,309 filed Apr. 5, 2007, which is herein incorporated by reference in its entirety.

Such doses may be desirably administered once a day to several times a day as divided portions. For example, the compositions of the present disclosure may be administered at least 1×, 2×, 3×, 4×, 5×, 6×, 8×, 10× or 20× a day. In certain embodiments the composition described herein is administered at least once a day for a period of days, weeks, months or years. The agent may be administered at least once, twice, three, or four times daily. Depending upon the desired therapeutic action, patient response and other factors, the dosage form may be administered between meals, during meals, prior to a meal (i.e., within 5, 10, 15, 20, 25, 30, 35, 40, 45, 50, 55, or 60 minutes, 2 hours, 4 hours, 8 hours, or 12 hours prior to eating) or after a meal (i.e., within 5, 10, 15, 20, 25, 30, 35, 40, 45, 50, 55, or 60 minutes, 2 hours, 4, hours, 8 hours, or 12 hours following a meal). In certain embodiments the dosage unit and daily dose are equivalent. In various embodiments, the dosage unit is administered with food at anytime of the day, without food at anytime of the day, with food after an overnight fast (e.g. with breakfast), at bedtime after a low fat snack.

### Kits

The compounds and pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions in the form of a label on the package or in the form of an insert included in the packaging of the kit. A kit can include additional components such as syringes or other means for administering the agents as well as diluents or other means for formulation.

Thus, the kits can comprise: a) a pharmaceutical composition comprising at least one sGC modulator and at least one lipid altering agent or PDE inhibitor and a pharmaceutically acceptable carrier, vehicle or diluent; and b) a container or packaging. The kits may optionally comprise instructions describing a method of using the pharmaceutical compositions in one or more of the methods described herein (e.g., preventing or treating dyslipidemia, hyperlipidemia, hypercholesterolemia, hypertriglyceridemia, sitosterolemia, familial hypercholesterolemia, xanthoma, combined hyperlipidemia, lecithin cholesterol acyltransferase deficiency, tangier disease, abetalipoproteinemia, erectile dysfunction, fatty liver disease, and hepatitis). The pharmaceutical composition comprising the at least one sGC modulator and at least one lipid altering agent or PDE inhibitor contained in the kit may be optionally combined in the same pharmaceutical composition.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory aid containing information and/or instructions for the physician, pharmacist or subject regarding when the medication is to be taken. A “daily dose” can be a single tablet or capsule or several tablets or capsules to be taken on a given day. When the kit contains separate compositions, a daily dose of one or more compositions of the kit can consist of one tablet or capsule while a daily dose of another one or more compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter that indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Various patent and/or scientific literature references have been referred to throughout this application. The disclosures of these publications in their entireties are hereby incorporated by reference as if written herein. In view of the above description and the examples below, one of ordinary skill in the art will be able to practice the disclosure as claimed without undue experimentation. The foregoing will be better understood with reference to the following Examples that detail certain procedures for the preparation of formulations according to the present disclosure. All references made to these Examples are for the purposes of illustration. The following Examples should not be considered exhaustive, but merely illustrative of only a few of the many embodiments contemplated by the present disclosure.

### Examples

Soluble guanylate cyclase modulators (e.g. nitroglycerin, isosorbide mononitrate) alone and in combination with other therapeutic agents can be tested for effects on lipid and cholesterol profiles in various lipid and cholesterol related animal models.

### Watanabe Rabbit Model

sGC modulators, alone and in combination, are tested for the ability to prevent or arrest atherosclerosis in a rabbit model of familial hypercholesterolemia. sGC modulators can be administered before or after onset of atherosclerosis for example as described in Havel et al. (1989) Arteriosclerosis 9:133-8, Aliev and Burnstock (1998) Histol Histopathol 13:797-817, Fan et al. (1999) Pathol Int. 49:583-94 and Brousseau and Hoeg (1999) J Lipid Res. 40:365-75.

### Rat High Fat Diet Model

sGC modulators, alone and in combination, are tested for effects on cholesterol and lipid profiles in a rat high fat diet model for example as described in Ghibaudi et al. (2002) Obes Res 10:956-63, Ricci and Levin (2003) Am J Physiol Regul Integr Comp Physiol 285:R610-8, and Gao et al. (2002) 936:87-90.

### Hamster High Fat Diet Model

sGC modulators, alone and in combination, are tested for effects on cholesterol and lipid profiles in a hamster high fat diet model for example as described in Wang et al. (2001) Eur J. Pharmacol 427:285-93 and van Heek et al. (2001) Diabetes 50:1330-5.

Although the foregoing disclosure has been described and depicted in terms of certain preferred embodiments, other specific embodiments may be effected by those skilled in the art to accomplish the same objectives and without departing from the true spirit of the scope of the present disclosure. Accordingly, the scope of the Applicant's disclosure is to be determined by reference to the attached claims, which are not limited to any of the particular embodiments disclosed herein.

We claim:

1. A method for preventing or treating lipid metabolism disorder, the method comprising administering to a patient in need thereof a therapeutically effective amount of a pharmaceutical composition comprising one or more soluble guanylate cyclase modulators.

2. The method of claim 1 wherein the one or more soluble guanylate cyclase modulators are chosen from NO donors, eNOS transcriptional enhancers, haem-dependent sGC stimulators, haem-independent sGC activators and non-arginine NOS substrates.

3. The method of claim 2 wherein the soluble guanylate cyclase modulator is an NO donor.

4. The method of claim 3 wherein the NO donor is chosen from organic nitrates, isosorbides, S-nitrosothiols, iron-nitrosyl complexes, sydnonimines, C-nitroso compounds, and secondary amine/NO complex ions.

5-10. (canceled)

11. The method of claim 2 wherein the soluble guanylate cyclase modulator is an eNOS transcriptional enhancer.

12. The method of claim 11 wherein the eNOS transcription enhancer is chosen from 2,2-difluorobenzo[1,3]dioxol-5-carboxylic acid indan-2-ylamide, 4-fluoro-N-(indan-2-yl)-benzamide), AVE3085 and AVE9488.

13. The method of claim 2 wherein the soluble guanylate cyclase modulator is a haem-dependent sGC stimulator.

14. The method of claim 13 wherein the haem-dependent sGC stimulator enhancer is chosen from YC-1, BAY 41-2272, BAY 41-8543, CFM-1571, and A350-619

15. The method of claim 2 wherein the soluble guanylate cyclase modulator is a haem-independent sGC activator.

16. The method of claim 15 wherein the haem-independent sGC activator is chosen from BAY 58-2667, HMR-1766, S 3448 (2-(4-chloro-phenylsulfonylamino)-4,5-dimethoxy-N-(4-(thiomorpholine-4-sulfonyl)-phenyl)-benzamide and HMR-1069.

17. The method of claim 2 wherein the soluble guanylate cyclase modulator is a non-arginine NOS substrate.

18. The method of claim 17 wherein the non-arginine NOS substrate is chosen from an n-hydroxyguanidine based analog, an L-arginine derivative, an N-alkyl-N′-hydroxyguanidine, an N-aryl-N′-hydroxyguanidine and a guanidine derivatives.

19. (canceled)

20. The method of claim 1 further comprising administering a therapeutically effective amount of at least one phosphodiesterase inhibitor or at least one lipid altering agent.

21-39. (canceled)

40. The method of claim 1 wherein the lipid metabolism disorder is chosen from dyslipidemia, hypercholesterolemia, hyperlipidemia, hypertriglyceridemia, sitosterolemia, and fatty liver disease.

41-112. (canceled)
